(19) United States
(12) Patent Application Publication
(10) Pub. No.: US 2021/0304871 A1
(43) Pub. Date: Sep. 30, 2021

(54) REMOTE HEALTHCARE MOBILE APPLICATION

(71) Applicant: Christopher Grey, Manhattan Beach, CA (US)

(72) Inventor: Christopher Grey, Manhattan Beach, CA (US)

(21) Appl. No.: 16/833,565

(22) Filed: Mar. 28, 2020

(51) Int. Cl. G16H 20/90; G16H 50/20; G16H 40/67; G16H 10/20

(52) U.S. Cl. CPC G16H 20/90; G16H 10/20; G16H 40/67; G16H 50/20

(57) ABSTRACT

A system and method that provides a Web-based mobile application for utilizing TCM, holistic medicine, and other healthcare expertise from a provider located a distance from the patient is disclosed.

### TECHNICAL FIELD

This application relates in general to a system and method for providing digital communications between remote healthcare providers, and more specifically, to a system and method for providing a mobile application for utilizing Traditional Chinese Medicine (TCM) and other healthcare expertise from a provider located a distance from the patient.

### BACKGROUND

Practitioners of traditional Chinese medicine and similar holistic medicine, especially those with certain expertise and skills, are not always available to patients located in remote areas or even patients located miles from the practitioner. It is critical that patients receive the best holistic healthcare possible, but distance and remote locations provide often insurmountable challenges to healthcare providers with in-demand expertise. In some cases, a local practitioner in remote areas even finds it difficult to communicate about a patient with a remote practitioner through telephone, fax, and other traditional methods.

Therefore, a need exists for a Web-based process that provides a mobile application for utilizing TCM and other healthcare expertise from a provider located a distance from the patient. Specifically, the mobile application is used to gather demographic and diagnostic information on potential patients in a single tool so that patients and healthcare workers in rural and less developed areas can obtain diagnostic assistance generally not available to them. The present invention attempts to address the existing limitations in a system and method for providing a mobile application for utilizing TCM and other healthcare expertise from a provider located a distance from the patient according to the principles and example embodiments disclosed herein.

### SUMMARY

In accordance with the present invention, the problems found in providing healthcare to rural and underdeveloped locales are solved by providing a system and method for a mobile application that utilizes TCM and other healthcare expertise from a provider located a distance from the patient according to the principles and example embodiments disclosed herein.

In one embodiment, the present invention is a system for providing a mobile application for utilizing holistic medicine and other healthcare expertise from a provider located a distance from the patient. This system permits rural patients and practitioners to diagnose via the Web and application possible medical conditions for early warning and prevention by submitting patient photos and data and related questions to a remote doctor or holistic medicine healthcare specialist.

In another embodiment, the present invention is a method for providing a mobile application for utilizing holistic medicine and other healthcare expertise from a provider located a distance from the patient. The great utility of the invention is that a system and method for providing a mobile application for utilizing holistic medicine and other healthcare expertise from a provider located a distance from the patient permits practitioners and patients to obtain care from experts typically not available to them.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages, will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment providing a system and method for a mobile application that utilizes TCM and other healthcare expertise from a provider located a distance from the patient according to the present invention.

FIG. 2a is a block diagram illustrating an exemplary hardware architecture of a computing device.

FIG. 2b is a block diagram illustrating an exemplary logical architecture for a client device.

FIG. 2c is a block diagram showing an exemplary architectural arrangement of clients, servers, and external services.

FIG. 2d is another block diagram illustrating an exemplary hardware architecture of a computing device.

FIGS. 3a-h illustrate example embodiments of user screen shots of a patient and practitioner mobile application that functions within a system and method providing a system and method for a mobile application that utilizes TCM and other healthcare expertise from a provider located a distance from the patient according to the present invention.

FIG. 4 illustrates a computing system of software components providing a mobile application that utilizes TCM and other healthcare expertise from a provider located a distance from the patient according to the present invention.

FIG. 5 illustrates a flowchart corresponding to a method performed by software components that provides a mobile application utilizing TCM and other healthcare expertise from a provider located a distance from the patient according to the present invention.

### DETAILED DESCRIPTION

This application relates in general to a system and method for a mobile application that utilizes TCM and other healthcare expertise from a provider located a distance from the patient according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 micrometers” should be interpreted to include not only the explicitly recited values of about 50 micrometers and 250 micrometers, but also include individual values and sub-ranges within the indicated range. Thus, included in this numerical range are individual values such as 60, 70, and 80 micrometers, and sub-ranges such as from 50-100 micrometers, from 100-200, and from 100-250 micrometers, etc.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

As used herein, the term “about” means that dimensions, sizes, formulations, parameters, shapes, and other quantities and characteristics are not, and need not, be exact, but may be approximated and/or larger or smaller as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The term “mobile application” refers to an application executing on a mobile device such as a smartphone, tablet, and/or web browser on any computing device.

The terms “healthcare practitioner” and “user” refer to an entity, e.g. a human, that offers TCM and other healthcare services directly to patients while obtaining assistance as needed from other providers remotely. In a particular case, user corresponds to a healthcare practitioner who uses a mobile application.

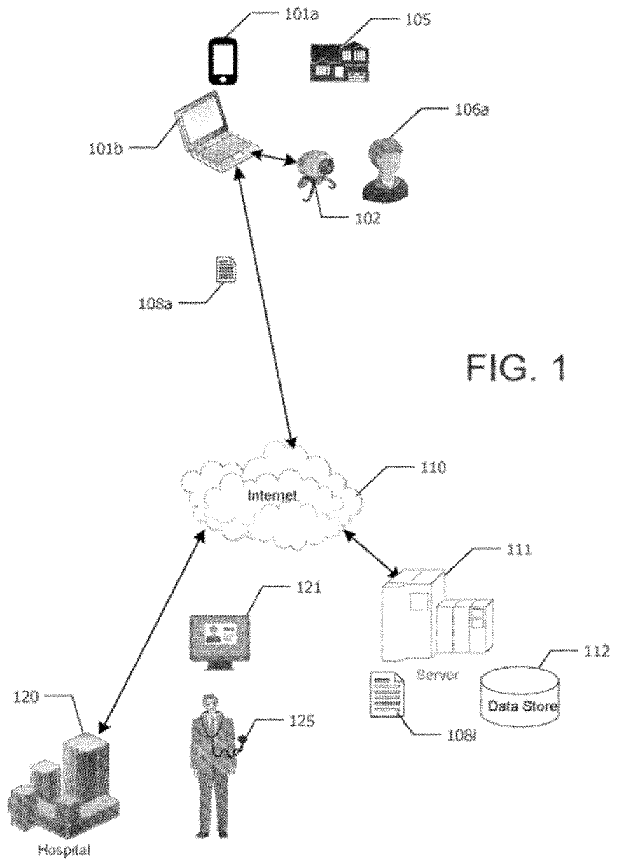

In general, the present disclosure relates to providing a system and method for a mobile application that utilizes TCM, holistic medicine, and other healthcare expertise from a provider located a distance from the patient. To better understand the present invention, FIG. 1 illustrates an example embodiment for a system for providing a mobile application that utilizes TCM, holistic medicine, and other healthcare expertise from a provider located a distance from the patient according to the present invention. A patient 106a uses a user device 101a-b to generate a patient data package 108 that contains data useful to a Traditional Chinese Medicine (TCM) practitioner 125. The patient data package 108 is transmitted to a web server 111 via the Internet 110 for retrieval and use by the TCM practitioner 125. The TCM practitioner 125 retrieves the patent data package 108 using a practitioner user device 121 to review, arrive at a diagnosis, and to generate treatment recommendations using TCM principles. The treatment recommendations are forwarded to the web server 111 for storage and forwarding to the user device 101a-b.

The Eight Principles of Traditional Chinese Medicine are:
1. Yin. Yin, along with yang is the most general classification for pattern diagnosis and it describes the relationship between the other three pairs of Principles. Generally speaking, Yin is cold.
2. Yang. Yang, along with yin is used to describe the relationship between the other three pairs of Principles. Typically Yang represents heat.
3. Interior. Interior describes diseases that manifest themselves in the zang-fu organs or deep inside the body, such as qi, blood, and bone marrow. More broadly, it's used to describe diseases that cannot be classified as Exterior.
4. Exterior. Exterior describes diseases that manifest themselves on surface of the body, such as hair, skin, nails, and meridians. Its clinical features include body chills, fever, aversion to cold temperatures and winds, a weak pulse, and headaches.
5. Heat. Heat describes the absence of an aversion to cold. If paired with an Exterior pattern, its symptoms can include a rapid pulse, fever, body chills, dehydration, and a sore throat. If paired with an Interior pattern, its symptoms can include a preference for cold drinks, clear urine, and a slow pulse.
6. Cold. Cold describes an aversion to cold. If paired with an Exterior pattern, its symptoms can include body aches, a tense pulse, fever, body chills, and headaches. If paired with an Interior pattern, its symptoms can include nausea, stomach pain, vomiting, and diarrhea.
1. Deficiency. Deficiency is used to describe a vacuity in qi, blood (xue), or body fluids Ginye). Depending on how it relates to Interior/Exterior and Cold/Heat, it can manifest as constipation, having a small appetite, dizziness, and slow pulse.
2. Excess. Excess is generally classified as any disease that can't be identified as a Deficiency pattern. Usually, it means that one of the Six Excesses is present. Depending on how it relates to Interior/Exterior and Cold/Heat, it can manifest as quick pulse, sweaty palms, and sharp stomach pains.

After a basic diagnosis is given via the Eight Principles, the diagnostic continues and focuses on more specific conditions. After evaluating the present symptoms, a person's condition is further evaluated as to how the specific entities (qi, meridians, zang-fu, etc.) are affected.

### Cause of Disease

Traditional Chinese medicine does not strongly differentiate between the cause and effect of a disease. However, there are three major categories that are considered to give rise to disease. The three major categories of disease causes are:
1. External: disease caused by the Six Excesses and bad qi.
2. Internal: disease caused the by Seven Emotions or Seven Affects: Anger, Fear, Anxiety, Fright, Griet: Pensiveness, and Joy. These are said to affect the zang-fu organs.
3. Other Irregularities: disease caused something that is neither external nor internal, such as having an unbalanced diet or damage done by parasites.

### Four J>inars of Diagnosis

There are four methods of diagnostics in TCM, which are often referred to as the Four Pillars of Diagnosis. They are:
1. Inspection. Inspection or “looking” focuses on visual analysis of the face, skin features, and particularly, the tongue. In TC:M, the surface of the tongue corresponds to particular zang-fil organs and can reveal a lot about a particular disease. Therefore, the tongue diagnosis is considered a cornerstone practice in the diagnostic process.
2. Auscultation. Auscultation refers to the analysis of particular sounds. In TCM, there are five major types of sounds: shouting, laughing, singing, weeping, and groaning. Auscultation analysis extends to olfaction, which analyzes the smell of body odor, even though sound and smell are considered to be separate senses in the \Vest.
3. Palpation. Palpation refers to analysis by feeling, particularly the, vlist pulse, abdomen, and meridian points. The techniques for doing this are complex and can take years to master.
4. Inquiry. Inquiry refers to analysis by asking questions about the person's past health and habits. Traditionally, this includes 10 questions, which range from diet to sleep pattern.

After recognizing a particular pattern of disharmony, a doctor will prescribe treatment based on the diagnosis that was given. The treatment can include the more common practices of TCM, such as herbal medicine, acupuncture, and tui na massage, but also less common practices, such as cupping.

The tongue has many relationships and connections in the body, both to the meridians and the internal organs. It is therefore very useful and important during inspection for confirming TCM diagnosis. It can present strong visual indicators of a person's overall harmony or disharmony. The tongue has a special relationship with the heart, in that the heart opens to the tongue. The tongue is said to be an “offshoot” of the heart, or “flowers” into the heart. The normal tongue in TCM has a light red or pinkish body with a thin white coating.

During tongue diagnosis, TCM practitioners usually examine the tongue coating, shape, and color. Tongue diagnosis also involves examining specific areas on the tongue. Here's a look at how these issues are addressed in a typical tongue diagnosis:

Color A light red color indicates that an individual's vital energy (also known as “qi”) is strong. Changes in tongue color, meanwhile, are said to signal chronic illness.

For example, pale coloring in the tongue is thought to indicate an issue with the pancreas and/or digestive function, while purple coloring is said to arise from blockages in the flow of the qi.

Shape, Normal tongue shape is neither too thick nor too thin; the tongue body is smooth with no cracks. In general, changes in tongue shape are thought to reflect chronic illness affecting the blood, bodily fluids, or qi.

Changes in tongue shape may include a swollen or puffy tongue which are believed to be another indicator of problems with the pancreas and/or digestive function, cracks in the tongue which are believed to be a possible sign of imbalance in the heart organ or an issue associated with insomnia and memory troubles, and curling at the sides of the tongue which is believed to indicate liver qi stagnation.

Coating Although the tongue coating is usually thin and white, a pale yellow and slightly thicker coating at the back of the tongue also may be normal.

In addition to reflecting the health of the spleen and stomach, tongue coating also provides an indication of acute illness, such as colds. For instance, a peeled or absent tongue coating may result from kidney yin deficiency, an issue associated with conditions like low back pain and tinnitus.

Currently, patients 106a who seek a health diagnosis from a TCM practitioner 107 in many rural and underdeveloped areas must see these providers in person. As noted above, TCM practitioner 107 needs an examination of patients to inspect a patient's tongue, examine a patient's back and front torso, and ask patients several questions about their general health status, including sleep timing, duration, and quality, diet, exercise, bowel movements and urination, energy level throughout the day, any soreness or pain in the body, level of pain and soreness, and mood and mental state, including anxiety, depression, and changes in temperament. Typically, rural and underdeveloped areas have few experienced TCM practitioners 107.

Additionally, patients find it very time consuming to visit certain TCM practitioners 107 in person because of required distances or difficult terrain that needs to be traveled, even if the patient can find a good practitioner. Currently remote diagnosis is hardly ever done for a number of reasons. Technology-based tools are just now being developed. Communications infrastructure has not been available in all rural and underdeveloped locations. Creation of a standardize set of patient data useful for a wide variety of diagnosis decisions has not been completed and agreed upon.

This remote diagnostic process replicates exactly what a TCM practitioner 107 does in person using Web and mobile software for uploading patient data and photos, and a form for answering submitted questions. Creating a patient data package 108a takes a patient 106 less than 5 minutes to complete. This patient data package 108a is then submitted to a TCM practitioner 107 who completes a standardized set of TCM diagnostic prescriptive responses to that patient data package 108a, also using web and mobile software, that is transmitted back to the patient 106 via the same software. Providing a diagnostic response may also take less than 5 minutes for the TCM practitioner 107 to complete. By allowing qualified, standardized, remote diagnosis by TCM practitioners 107, potentially millions of people will have access to important early warning and preventive health recommendations. Use of this application and process may lead to healthier lifestyle choices and healthcare decisions for many millions of people.

While all of the above functions are described to be provided to users via a mobile application on a smartphone, one of ordinary skill will recognize that any computing device including tablets, laptops, and general purpose computing devices also may be used. In at least one embodiment, all of the services described herein are provided using Web pages accessed from a Web server 111 using a Web browser such as Safari™, Firefox™, Chrome™, DuckDuckGo™, and the like. All of the screen examples described herein show user interface elements that provide the functionality of the present invention. The arrangement, organization, presentation, and use of particular user input/output (I/O) elements including hyperlinks, buttons, text fields, scrolling lists, and similar I/O elements are shown herein for example embodiments only to more easily convey the features of the present invention. The scope of the present invention should not be interpreted as being limited by any of these elements unless expressly recited within the attached claims.

For the purpose of the example embodiment of FIG. 1, various functions are shown to be performed on different programmable computing devices that communicate with each other over the Internet 105. These computing devices may include smartphones 101a, laptop computers 101b, tablets (not shown), and similar devices so long as the disclosed functionality of the mobile application described herein is supported by the particular computing device. One of ordinary skill will recognize that this functionality is grouped as shown in the embodiment for clarity of description. Two or more of the processing functions may be combined onto a single processing machine. Additionally, it may be possible to move a subset of processing from one of the processing systems shown here and retain the functionality of the present invention. The attached claims recite any required combination of functionality onto a single machine, if required, and all example embodiments are for descriptive purposes.

For all of the above devices that are in communication with each other, some or all of them need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects, and in order to more fully illustrate one or more aspects. Similarly, although process steps, method steps, algorithms or the like may be described in a sequential order, such processes, methods, and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method or algorithm is carried out or executed. Some steps may be omitted in some aspect or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the aspects disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific aspects, at least some of the features or functionalities of the various aspects disclosed herein may be implemented on one or more general-purpose computers associated with one or more networks, such as for example an end-user computer system, a client computer, a network server or other server system, a mobile computing device (e.g., tablet computing device, mobile phone, smartphone, laptop or other appropriate computing device), a consumer electronic device, a music player or any other suitable electronic device, router, switch or other suitable device, or any combination thereof. In at least some aspects, at least some of the features or functionalities of the various aspects disclosed herein may be implemented in one or more virtualized computing environments (e.g., network computing clouds, virtual machines hosted on one or more physical computing machines or other appropriate virtual environments).

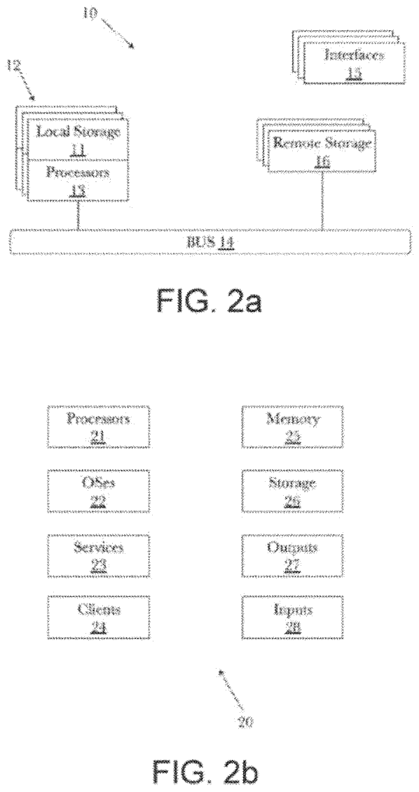

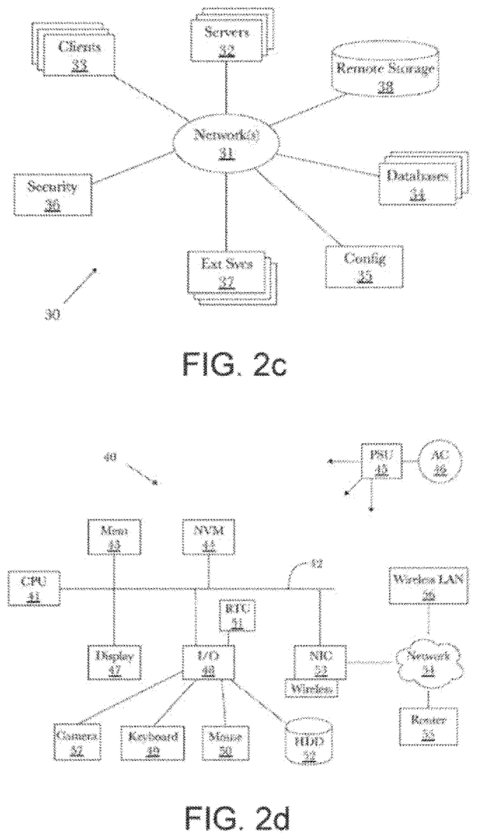

Referring now to FIG. 2a, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network, a metropolitan area network, a local area network, a wireless network, the Internet or any other network, using known protocols for such communication, whether wireless or wired.

In one aspect, computing device 10 includes one or more central processing units (CPU) 12, one or more interfaces 15, and one or more buses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing a CPU 12, local memory 11 and/or remote memory 16, and interface(s) 15. In at least one aspect, a CPU 12 may be caused to perform one or more of the different types of functions and/or operations under the control of software modules or components, which for example, may include an operating system and any appropriate applications software, drivers, and the like.

A CPU 12 may include one or more processors 13 such as, for example, a processor from one of the Intel, ARM, Qualcomm, and AMD families of microprocessors. In some aspect, processors 13 may include specially designed hardware such as application-specific integrated circuits (ASICs), electrically erasable programmable read-only memories (EEPROMs), field-programmable gate arrays (FPGAs), and so forth, for controlling operations of a computing device 10. In a particular aspect, a local memory 11 (such as non-volatile random access memory (RAM) and/or read-only memory (ROM), including for example one or more levels of cached memory) may also form part of a CPU 12. However, there are many different ways in which memory may be coupled to a system 10. Memory 11 may be used for a variety of purposes such as, for example, caching and/or storing data, programming instructions, and the like. It should be further appreciated that a CPU 12 may be one of a variety of system-on-a-chip-(SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may, for example, support other peripherals used with a computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, gigabit Ethernet interfaces, serial ATA (SATA) or external SATA (ESATA) interfaces, high-definition multimedia interfaces (HDMI), digital visual interfaces (DVI), analog or digital audio interfaces, asynchronous transfer mode (ATM) interfaces, high-speed serial interfaces (HSSI), point of sale (POS) interfaces, fiber data distributed interfaces (FDDis), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 2a illustrates one specific architecture for a computing device 10 for implementing one or more of the aspects described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, a single processor 13 handles communications as well as routing computations, while in other aspects a separate, dedicated communications processor may be provided. In various aspects, different types of features or functionalities may be implemented in a system according to the aspect that includes a client device (such as a tablet or smartphone running client software) and a server system (such as a server system described in more detail below).

Regardless of network device configuration, the system of an aspect may employ one or more memories or memory modules (for example, remote memory block 16 and local memory 11) configured to store data, program instructions for the general-purpose network operations or other information relating to the functionality of the aspects described herein (or any combinations of the above). Program instructions may control execution of or comprise an operating system and/or one or more applications, for example. Memory 16 or memories 11, 16 may also be configured to store data structures, configuration data, encryption data, historical system operations information or any other specific or generic non-program information described herein.

Because such information and program instructions may be employed to implement one or more systems or methods described herein, at least some network device aspects may include nontransitory machine-readable storage media, which, for example, may be configured or designed to store program instructions, state information, and the like for performing various operations described herein. Examples of such nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device) or they may be removable such as swappable flash memory modules (such as “thumb drives” or other removable media designed for rapidly exchanging physical storage devices), “hot-swappable” hard disk drives or solid state drives, removable optical storage disks, or other such removable media, and that such integral and removable storage media may be utilized interchangeably.

Examples of program instructions include both object code, such as may be produced by a compiler, machine code, such as may be produced by an assembler or a linker, byte code, such as may be generated by for example by a Java™ compiler and may be executed using a Java virtual machine or equivalent, or files containing higher level code that may be executed by the computer using an interpreter (for example, scripts written in Python™, Perl™, Ruby™, Groovy™, or any other scripting language).

In some aspects, systems may be implemented on a standalone computing system. Referring now to FIG. 2b, there is shown a block diagram depicting a typical exemplary architecture of one or more aspects or components thereof on a standalone computing system. A computing device 20 includes processors 21 that may run software that carry out one or more functions or applications of aspects, such as for example a client application 24. Processors 21 may carry out computing instructions under control of an operating system 22 such as, for example, a version of MICROSOFT WINDOWS™ operating system, APPLE macOS™ or iOS™ operating systems, some variety of the LINUX™ operating system, ANDROID™ operating system, or the like. In many cases, one or more shared services 23 may be operable in system 20, and may be useful for providing common services to client applications 24. Services 23 may, for example, be WINDOWS™ services, user-space common services in a LINUX™ environment or any other type of common service architecture used with an operating system 21. Input devices 28 may be of any type suitable for receiving user input including, for example, a keyboard, touchscreen, microphone (for example, for voice input), mouse, touchpad, trackball or any combination thereof. Output devices 27 may be of any type suitable for providing output to one or more users, whether remote or local to a system 20, and may include, for example, one or more screens for visual output, speakers, printers or any combination thereof. Memory 25 may be RAM having any structure and architecture known in the art, for use by processors 21, for example to run software. Storage devices 26 may be any magnetic, optical, mechanical, memristor or electrical storage device for storage of data in digital form (such as those described above, referring to FIG. 2a). Examples of storage devices 26 include flash memory, magnetic hard drive, CD-ROM, and the like.

In some aspects, systems may be implemented on a distributed computing network, such as one having any number of clients and/or servers. Referring now to FIG. 2c, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in Fig. B. In addition, any number of servers 32 may be provided for handling requests received from one or more clients 33. Clients 33 and servers 32 may communicate with one another via one or more electronic networks 31, which may be in various aspects any Internet, wide area network, mobile telephony network (such as CDMA or GSM cellular networks), wireless network (such as WiFi, WiMAX, LTE, and so forth) or local area network (or indeed any network topology known in the art; the aspect does not prefer any one network topology over another). Networks 31 may be implemented using any known network protocols, including, for example, wired and/or wireless protocols.

In addition, in some aspects, servers 32 may call external services 37 when needed to obtain additional information or to refer to additional data concerning a particular call. Communications with external services 37 may take place, for example, via one or more networks 31. In various aspects, external services 37 may comprise Web-enabled services or functionality related to or installed on the hardware device itself. For example, in one aspect where client applications 24 are implemented on a smartphone or other electronic device, client applications 24 may obtain information stored on a server system 32 in the Cloud or on an external service 37 deployed on one or more of a particular enterprise's or user's premises. In addition to local storage on servers 32, remote storage 38 may be accessible through the network(s) 31.

In some aspects, clients 33 or servers 32 (or both) may make use of one or more specialized services or appliances that may be deployed locally or remotely across one or more networks 31. For example, one or more databases 34 in either local or remote storage 38 may be used or referred to by one or more aspects. It should be understood by one having ordinary skill in the art that databases in storage 34 may be arranged in a wide variety of architectures and use a wide variety of data access and manipulation means. For example, in various aspects one or more databases in storage 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some aspects, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system or a logical database within an overall database management system. Unless a specific meaning is specified for a given use of the term “database,” it should be construed to mean any of these senses of the word, all of which are understood as a plain meaning of the term “database” by those having ordinary skill in the art.

Similarly, some aspects may make use of one or more security systems 36 and configuration systems 35. Security and configuration management are common information technology (IT) and web functions, and some amount of each are generally associated with any IT or web system. It should be understood by one having ordinary skill in the art that any configuration or security subsystems known in the art now or in the future may be used in conjunction with aspects without limitation, unless a specific security 36 or configuration system 35 or approach is required by the description of any specific aspect.

FIG. 2d shows an exemplary overview of a computer system 40 as may be used in any of the various locations throughout the system. It is exemplary of any computer that may execute code to process data. Various modifications and changes may be made to a computer system 40 without departing from the broader scope of the system and method disclosed herein. A CPU 41 is connected to a bus 42, to which bus is also connected to memory 43, nonvolatile memory 44, display 47, I/O unit 48, and network interface card (NIC) 53. An I/O unit 48, typically, may be connected to peripherals such as a keyboard 49, pointing device 50, hard disk 52, real-time clock 51, camera 57, and other peripheral devices. A NIC 53 connects to a network 54, which may be the Internet or a local network, which local network may or may not have connections to the Internet. The system may be connected to other computing devices through the network via a router 55, wireless local area network 56 or any other network connection. Also shown as part of a system 40 is a power supply unit 45 connected, in this example, to a main alternating current (AC) supply 46. Not shown are batteries that could be present and many other devices and modifications that are well known, but are not applicable, to the specific novel functions of the current system and method disclosed herein.

It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various aspects, functionality for implementing systems or methods of various aspects may be distributed among any number of client and/or server components. For example, various software modules may be implemented for performing various functions in connection with the system of any particular aspect, and such modules may be implemented to run on server and/or client components.

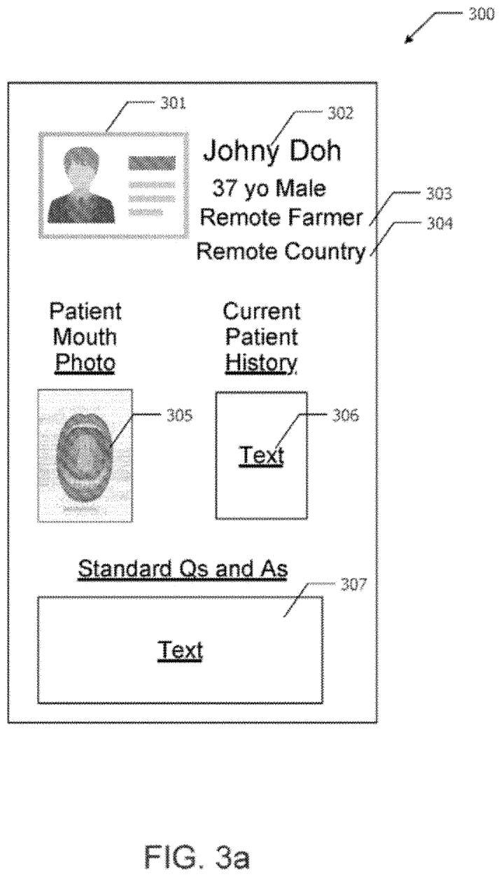

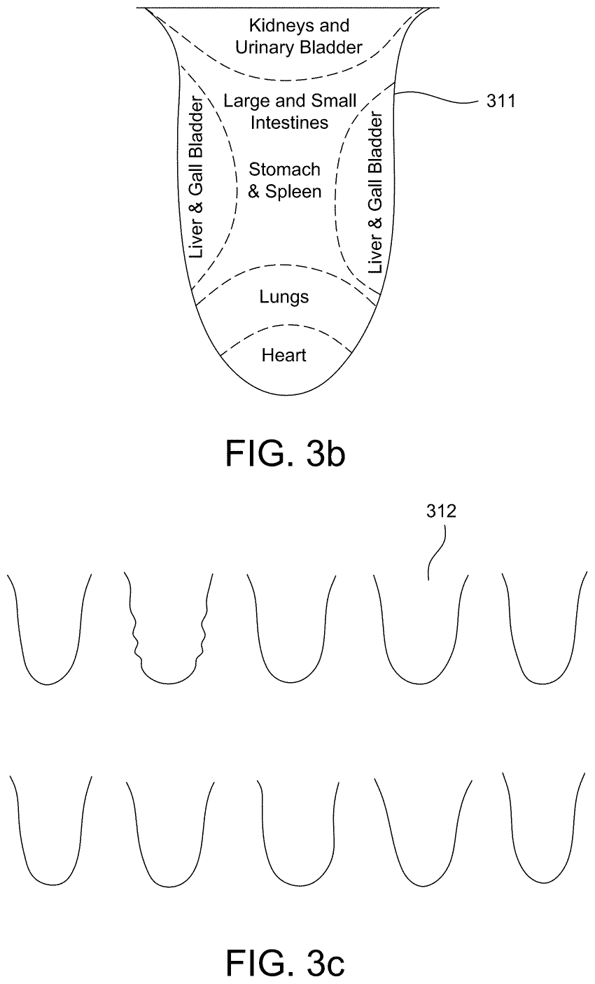

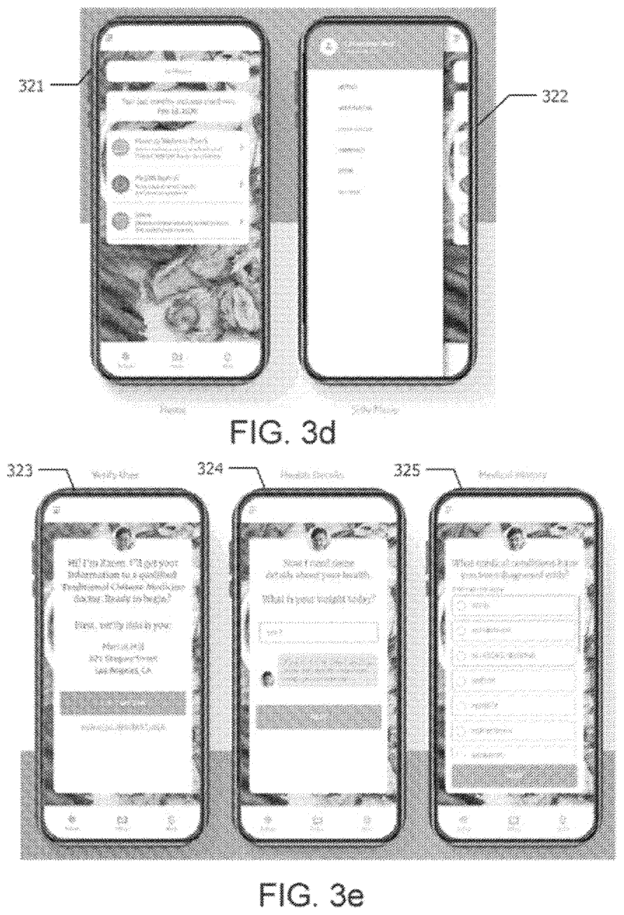

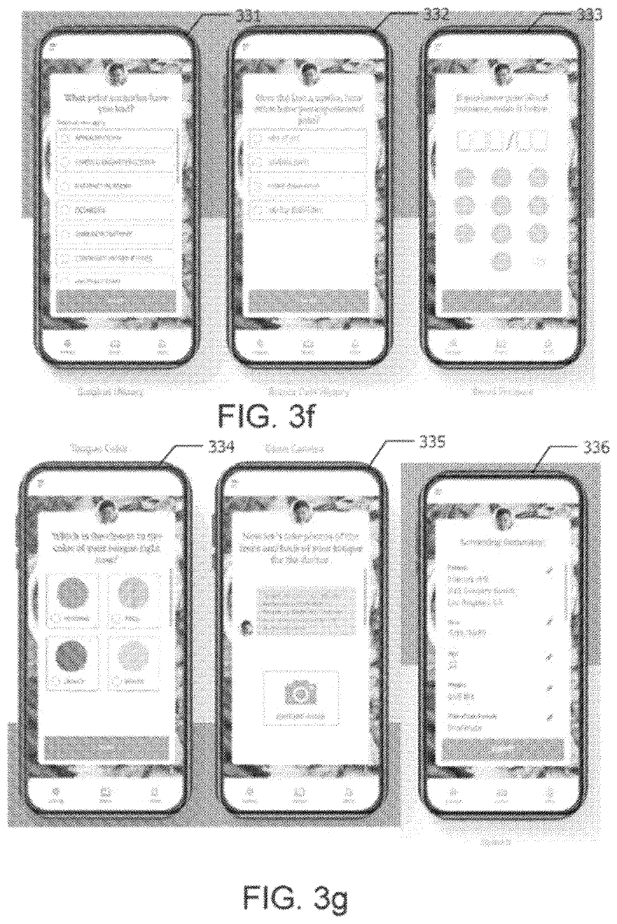

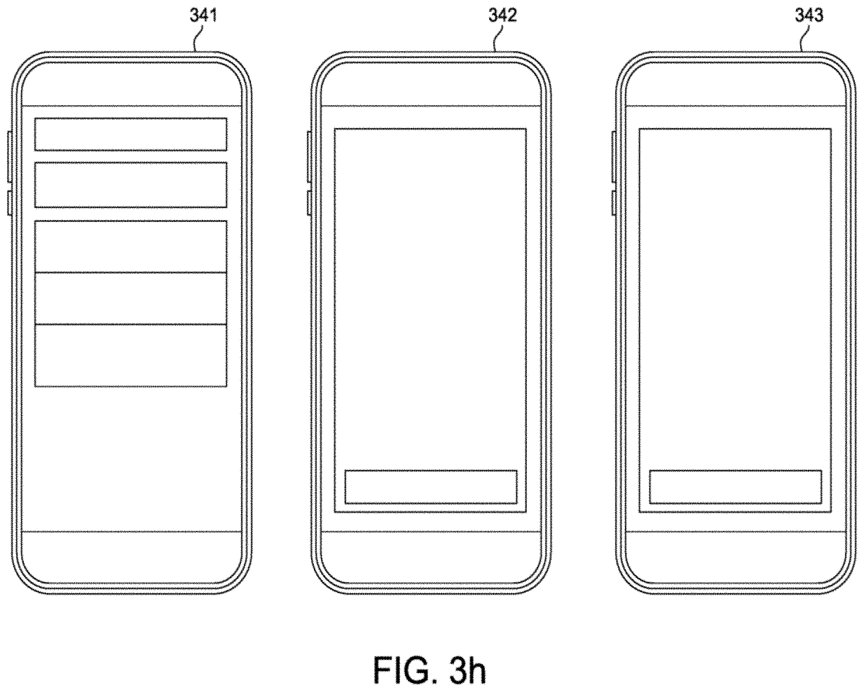

FIGS. 3a-h illustrate example embodiments of user screen shots of a patient and practitioner mobile application that functions within a system and method for providing TCM and other healthcare expertise from a provider located a distance from the patient according to the present invention. FIG. 3a illustrates a patient data package 300 typically comprised of all necessary data about the patient that is known or may be obtained at a remote location. This patient data package 300 may include patient name 302, age and occupation 303, and location 304. The patient data package 300 may also provide image data on a current patient's mouth, including both front and back of the patient's tongue 305, current patient history 306 for all past illnesses, treatments, and medicines being taken, and answers to a set of standard questions presented for completion based upon the complaints from the patient 106. A default set of questions may be used in general cases where a separate set of questions or an additional set of questions may be provided based upon current complaints and circumstances.

For example, questions related to respiratory symptoms may be asked when shortness of breath is a complaint. Questions may be tailored to local circumstances if a remote region has experienced a particular disease outbreak or the residents of the region have been exposed to particular contaminants or natural disasters.

Example questions that are typically requested for a patient 106 to provide as part of a patient data package 300 include:
1. Age, sex, height & weight
2. Medical history and surgeries if any
3. Sleep condition—Is it easy to fall asleep? Are you able to sleep through the night? If not, what time do you wake up or have any dreams?
4. Energy level—Do you feel tired when first wakeup? Do you feel tired during the day? Do you find it easy to concentrate?
5. Sweat—Do you find it easy to sweat? Do you have night sweats? Do you hardly sweat?
Are you having non-stop sweating?
6. Appetite—Do you have a good or a bad appetite? Do you have any cravings? How many meals a day do you eat?
7. Bowel movement—Are you constipated? Do you have any diarrhea? Does the bowel movement stink? What form does the bowel movement present? How many times a day do you have one? What is its color?
8. Urination—What color is your urine? How many times a day do you urinate? Do you have any night urination? Do you have trouble urinating or have any urgency?
9. Feeling cold or hot?—Are your hands and feet cold? Are you afraid of coldness or heat? Compare temperature differences between your forehead, hands, and feet. What shower temperature do you prefer?
10. Thirst-Do you feel thirsty? Do you prefer to drink hot, warm, room temp, or ice water?
11. Pain—Do you feel pain, and if so, in which area? What frequency do you have this pain? Do you prefer massage or a heat pad on the pain area? tightness?
12. Mental condition—Do you have a short temper? Do you feel you are depressed? Do you feel any anxiety? Do you feel happy? Do you worry? Are you ever impulsive? Do you find it hard to concentrate?
13. Diet—what is your preference of food? Do you drink coffee or alcohol? Do you eat sweets?
14. Sexual function—For a male, Do you have an erection in the morning? For a female, do you have any breast sensitivity in the morning? Do you obtain sexual satisfaction?
15. For females only—Detailed description of your period, its frequency, and its color? What is a typical length for your period? Is the bleeding heavy or light? Does the bleeding include black clots? Do you feel significant pain during your period? Have you ever been pregnant? Have you started menopause? Do you suffer from PMS?
16. Please submit photos of tongue—One from the front, one from a 45 degree angle, and one with the tongue rolled-up. When taking the tongue photos, don't use a flash, keep a distance of 15 cm, and take photos under regular light. Please avoid any yellow spotlight. Also, please put a piece of white paper next to the tongue for easy comparison.
17. Complaints—What are the main issues that you prefer to treat first?
18. Flu or cold?—Please provide a detailed description of fever, cough, mucus, or stuffy nose. Describe any headaches or nausea. Please describe any GI issues, pain, sweating, or exhaustion. Have you recently taken any Western meds or had an IV?

The questions request that photographs of the patient and his/her observable condition be obtained and included within the patient data package. If a mobile device such as a laptop or smartphone 101a-b has an internal camera, the images may be easily obtained by the mobile application. A separate camera 102 connected to the computing devices may also obtain images as needed with these images being uploaded to the mobile application for inclusion within the patient data package 108a.

FIGS. 3b-c illustrate example tongue areas and colors used by TCM practitioners that are typically compared with photographs received from a patient 106 as part of a patient data package 300. Areas of interest on a tongue 311 are shown in FIG. 3b. Tongues 312 having different colors related to various diagnoses are shown in FIG. 3c.

FIGS. 3d-e show various example user screens for a mobile application used by a patient 106 according to the present invention. These screens include a home screen 321, a side menu screen 322, a verify user screen 323 to confirm the identity of a patient 106, a health detail screen 324 for obtaining specific health complaints and symptoms from the patient 106, and a medical history screen 325 for obtaining any new medical history data from the patient.

FIGS. 3f-g show various additional example user screens for a mobile application used by a patient 106 according to the present invention. These screens include a surgical history screen 331 for obtaining any new surgical history data from the patient, a recent pain screen 332 for obtaining a description of any new surgical history data from the patient, a blood pressure screen 333 for obtaining any available blood pressure data from the patient, a self-exam tongue color description screen 334 for obtaining a patient's estimate for a current color of his or her tongue, an open camera screen 335 for obtaining photographs of the patient and his or her mouth and tongue, and a summary screen 336 showing all of the data entered by the patient 106.

FIG. 3h shows various example practitioner screens for a mobile application used by a TCM practitioner 107 according to the present invention. These screen shots include a practitioner home screen 341, a patient report screen 342, and a patient diagnosis screen 343.

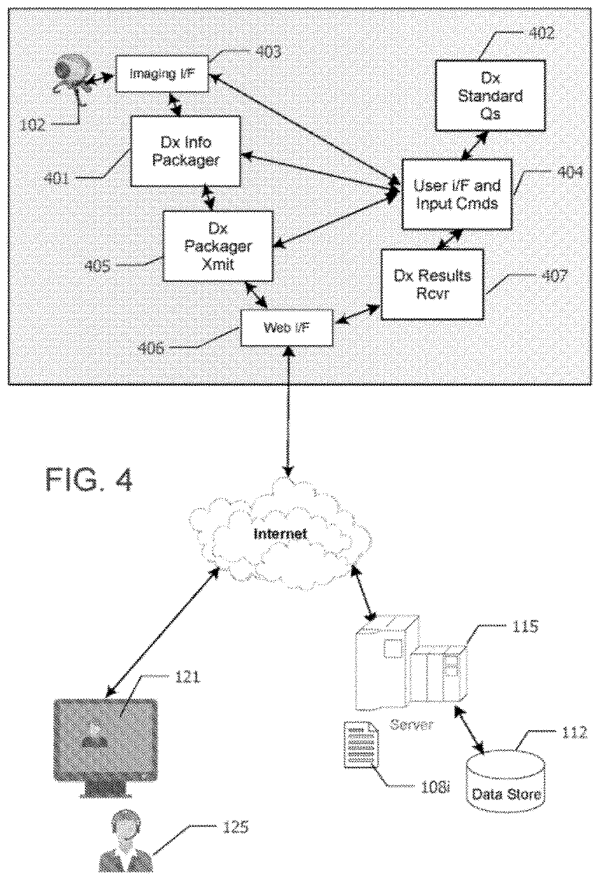

FIG. 4 illustrates a computing system of software components providing TCM and other healthcare expertise from a provider located a distance from the patient according to the present invention. A mobile application 400 that implements the disclosed functions may be constructed using a plurality of interrelated computing elements. These computing elements may be software-based computing processes executing on a programmable device. These computing elements may also be a hardware-based or firmware-based computing element that may be constructed to implement each element. One of ordinary skill in the art will recognize the ability for these computing elements to be constructed using one or more of these implementation technologies as desired.

In this particular embodiment, the mobile application 400 includes a user interface and input command processor 403, a web interface module 406, an imaging interface 403 coupled to a camera or imaging device 102, a diagnostic data packager 401, a diagnostic data package transmitter 405, a diagnostic data response receiver 407, and a datastore of diagnostic questions 402 used when creating a patient data package. The user interface and input command processor 403 acts as a control processor that interacts with a user and processes any commands initiated by the user. The user interface and input command processor 403 may forward a series of commands and supporting data to other computing elements as needed to accomplish a task.

The Web interface module 406 provides a mechanism for the mobile application 400 to connect to and communicate with other devices via the Internet 105. The Web interface module 406 provides all of the necessary communication protocol functions and data formatting to support physical landline connections to the Internet 105 as well as wireless networking connections including WiFi, cellular, satellite, and related communication methodologies.

An imaging interface module 403 may be coupled to a camera or imaging device 102 to obtain and upload images of the patient 106. This imaging interface module 403 may perform the image capture and upload functions, as well as any image format transformations needed to create a patient data package in a supported format. This imaging interface 403 may also allow images captured to be processed to adjust image intensity and color levels and other ordinary image processing operations that may improve images for inclusion within the patient data package.

The diagnostic data packager 401 obtains all of the data to be included within the patient data package and formats it into a standardized format for sharing with other healthcare providers. The diagnostic data packager 401 obtains image data of at least the top and bottom of the patient's tongue from the imaging interface module 403. The diagnostic data packager 401 obtains answers to questions from the datastore of questions 402 that are entered into the mobile application 400 by a user. Other data from any other available data source may also provide data to be included within the patient data package as well. The diagnostic data packager 401 combines all of the data from these various sources into a single data item. Data compression may also be performed on the data by the packager 401 if desired.

The diagnostic data package transmitter 405 is responsible for sending a completed patient data package received from the diagnostic data packager 401 to the remote healthcare provider 125 when commanded by a user command received from the user interface and input command processor 404. Any scheduling of data transmission to conform to operating schedules and particular remote destinations is performed as may be appropriate. For example, if a patient data package is marked urgent as opposed to routine, a different healthcare provider and location may be chosen based on the time of day, the symptom or complaint or other factors if particular needs for a given patient must be met.

The diagnostic data response receiver 407 is responsible for receiving data package responses from remote healthcare providers and informing the local user of the mobile application of the response. The diagnostic data response receiver 407 unpacks and decompresses the received data as needed in order for the response to be viewable when presented to the user via the user interface and input command processor 404. The diagnostic data response receiver 407 may also generate notifications to the mobile application 400 user of the arrival of a response and of the continuing existence of unread responses having a high or increasing priority based upon the patient's complaints and/or the particular contents of the response. These responses may also be stored for later retrieval on a remote Web server 115 by the diagnostic data response receiver 407.

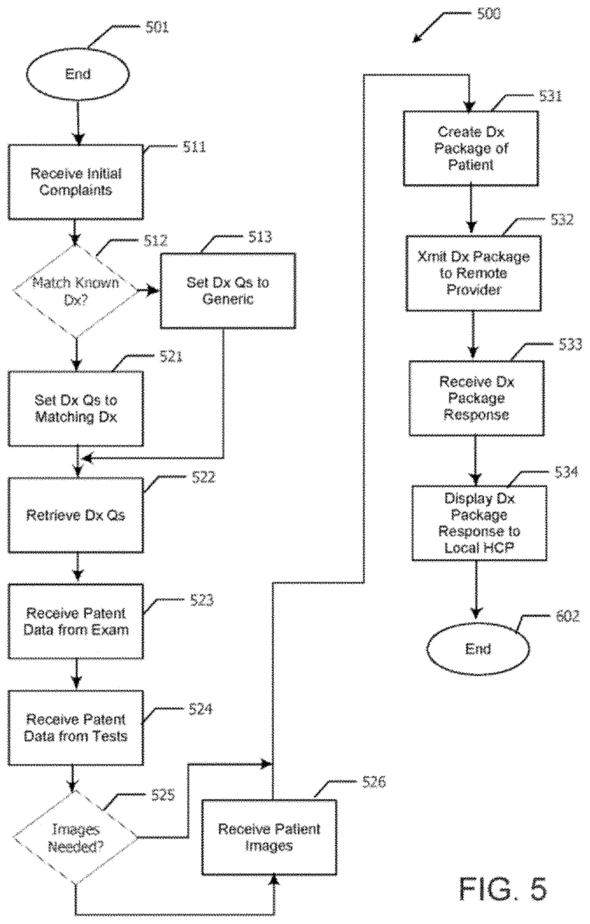

FIG. 5 illustrates a flowchart corresponding to a method performed by software components for providing TCM and other healthcare expertise from a provider located a distance from the patient according to the present invention. The process begins 501 when initial patient complaints or conditions are received by the mobile application 400 in step 511.

Optional test step 512 may determine whether condition-specific questions should be included in a patient data package. If a test step determines that condition-specific questions are not necessary, step 513 identifies generic or default questions to be retrieved by step 522.

When a test step determines that condition-specific questions are needed, step 521 identifies specific questions in place of or in addition to a set of generic or default questions to be retrieved by step 522. Once the needed questions have been identified, step 522 retrieves the questions from a question datastore 402 to be presented to the user for completion using data obtained from the patient. The user enters the patient data into the mobile application 400 in step 523. Other patient data, such as exam data or other test results, may be entered in step 524.

Test step 525 determines whether images are needed from the patient and if so, the needed images, including both front and back images of the patient's tongue, are compiled in step 526. Otherwise, the processing continues to step 531 in which all of the received patient data and images are combined into a single entity representing a patient data package. The patient data package is transmitted to a remote healthcare practitioner in step 532.

When a response to a submission of a patient data package is received, step 533 receives the response data and unpacks it as appropriate. The unpacked response data is presented to the user of the mobile application 400 in step 534 for use as appropriate and the process ends 502.

The mobile application may provide notification of all pending responses, using notification functions supported by operating systems for smartphones, tablets, and personal computers. These notifications may also be used to notify users of other actions.

For example, a user may receive a notification that a response to a Dx patient data package is due or has been received from a remote provider. When the user logs into the mobile application, the notification is provided and, if selected by the user, the corresponding Dx package response is shown so the user may take appropriate action using the mobile application. Healthcare professionals and patients also may receive notification of messages or other communications that may require immediate responses.

Additionally, the embodiments described herein are implemented as logical operations performed by a computer. The logical operations of these various embodiments of the present invention are implemented (1) as a sequence of computer-implemented steps or program modules running on a computing system and/or (2) as interconnected machine modules or hardware logic within the computing system. The implementation is a matter of choice dependent on the performance requirements of the computing system implementing the invention. Accordingly, the logical operations making up the embodiments of the invention described herein can be variously referred to as operations, steps or modules.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps where the additional, unrecited elements or steps do not have the one or more specified characteristics.

What is claimed is:

1. A system for providing remote holistic medicine healthcare diagnosis, the system comprising:
a user device for receiving patient input data, the user device comprises:
an imaging interface coupled to a camera or imaging device;
a diagnostic data packager;
a diagnostic data package transmitter; and
a datastore of diagnostic questions used when creating a patient data package;
a web server for maintaining records regarding patients and for communicating with health care practitioners;
wherein the patient data package comprises:
an image of a top of a patient's tongue;
an image of a bottom of the patient's tongue; and
a plurality of answers to diagnostic questions from the datastore of diagnostic questions.

2. The system according to claim 1, wherein the system further comprises:
a user interface and input command processor;
a web interface module; and
a diagnostic data response receiver.

3. The system according to claim 1, wherein the diagnostic questions from the datastore of diagnostic question correspond to inquiries related to traditional Chinese medicine diagnosis and treatment.

4. The system according to claim 1, wherein the system further comprises:
a practitioner user device for receiving the patent data package from the web server and generating a diagnosis and treatment recommendations.

5. The system according to claim 4, wherein the web server maintains all communications with patient user devices, practitioner user devices, patient data packages, and diagnosis and treatment recommendations.

6. A method for providing remote holistic medicine diagnosis, the method comprising:
generating a patient data package, the patient data package comprising:
image data of a top of a patient's tongue;
image data of a bottom of a patient's tongue;
a plurality of answers to diagnostic questions from a datastore of diagnostic questions;
transmitting the patent data package to a web server; and
receiving a diagnosis and recommended treatment from a TCM practitioner generated using TCM principles.

7. The method according to claim 6, wherein the method further comprises:
receiving image data of a top of a patient's tongue;
receiving image data of a bottom of a patient's tongue;
receiving a plurality of answers to diagnostic questions from the datastore of diagnostic questions.

8. The method according to claim 6, wherein the TCM practitioner retrieves the patient data package from the web server to generate the diagnosis and recommended treatment.

9. The method according to claim 8, wherein the TCM practitioner transmits the diagnosis and recommended treatment to the web server for access by the user device.

10. The method according to claim 6, wherein the web server maintains all communications with patient user devices, practitioner user devices, patient data packages, and diagnosis and treatment recommendations.
